(19) United States
(12) Patent Application Publication
(10) Pub. No.: US 2005/0020541 A1
(43) Pub. Date: Jan. 27, 2005

(54) Silicon compounds

(76) Inventors: Reinhold Tacke, Eisingen (DE); Jurgen Daiss, Wurzburg (DE)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/493,977

(22) PCT Filed: Oct. 30, 2002
(86) PCT No.: PCT/GB02/04900

(30) Foreign Application Priority Data
Oct. 30, 2001 (GB) 0126036.3

(51) Int. Cl. A61K 31/695; C07F 7/08

(52) U.S. Cl. 514/63; 556/406

(57) ABSTRACT

A compound of formula (I): wherein R¹ and R² are, independently, hydrogen or alkyl or together, with the nitrogen atom, form a heterocyclyl; R³ and R⁴ are, independently, hydrogen, hydroxyl, alkyl, alkoxy, alkanoyloxy, cyano, nitro, alkylmercapto, amino, alkylamino, dialkylamino, alkanamido, halogen or trifluoromethyl; R⁵ is hydrogen or alkyl; and n is 0, 1, 2, 3 or 4; or a pharmaceutically acceptable salt thereof or a prodrug form that is hydrolysable to a compound as defined above.

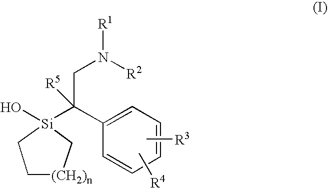

### FIELD OF THE INVENTION

This invention relates to compounds and their therapeutic use.

### BACKGROUND OF THE INVENTION

Noradrenaline, 5-hydroxytryptamine (5-HT, serotonin) and dopamine are mammalian monoamine neurotransmitters.

Noradrenaline (norepinephrine) acts as a neurotransmitter in the sympathetic nervous system and as a hormone throughout the body. Its neurotransmitter effects include regulation of mood, whilst its hormone effects include the control of blood pressure, heart rate, breathing and contraction of the gastrointestinal tract.

5-HT is widely distributed throughout the body, including blood platelets, intestinal wall and the central nervous system (CNS). 5-HT plays a role in inflammatory responses similar to histamine. It also acts as a neurotransmitter in the CNS, playing a role in mood control. Dopamine is a catecholamine, and acts on dopamine and adrenergic receptors throughout the body. It also stimulates the release of noradrenaline from nerve endings. Dopamine affects brain processes that control movement, emotional response and the ability to experience pleasure and pain. Dopamine has been implicated substantially in Parkinson's Disease and also plays a role in addiction.

Compounds that selectively modulate the activity of these neurotransmitters, either individually or in any combination, may serve as effective therapeutic agents for the treatment of a wide variety of diseases of the central or peripheral nervous systems. For example, the mechanisms involved in the generation of chronic pain syndromes such as neuropathic pain are not well understood, but supraspinal and spinal events, which modulate nociceptive transmission from the periphery to the CNS, could be mediated by 5-HT and noradrenaline pathways. 5-HT pathways are also thought to play a role in modulation of endorphin effects. These monoamines may therefore play an important role in transmission of chronic pain signals.

Venlafaxine, i.e. 1-[2-dimethylamino-1-(4-methoxyphenyl)ethyl]cyclohexan-1-ol, is an antidepressant drug, the preparation of which is disclosed in U.S. Pat. No. 4,535,186. A review of its pharmacology and clinical efficacy is contained in Montgomery, J. Clin. Psychiatry, 54, 119-126 (1993). Venlafaxine is a serotonin/noradrenaline reuptake inhibitor. There are, however, side-effects associated with its use as a medicament, including nausea, insomnia, headache, dizziness, sweating and occasionally convulsions.

Sila-substitution (C/Si-exchange) of drugs is a relatively recent approach for searching for organosilicon compounds which have beneficial biological properties. The approach involves the replacement of specific carbon atoms in compounds by silicon, and monitoring how the biological properties of the compounds have changed. A review of this approach is provided in Tacke and Zilch, Endeavour, New Series, 10, 191-197 (1986).

### SUMMARY OF THE INVENTION

The present invention provides compounds containing a silicon atom and which have desirable properties.

Compounds of the invention are of formula I: 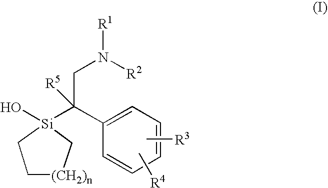
wherein R¹ and R² are, independently, hydrogen or alkyl or together, with the nitrogen atom, form a heterocyclic ring;
R³ and R⁴ are, independently, hydrogen, hydroxyl, C₁₋₆ alkyl, alkoxy, alkanoyloxy, cyano, nitro, alkylmercapto, amino, alkylamino, dialkylamino, alkanamido, halogen or trifluoromethyl;
R⁵ is hydrogen or alkyl; and
n is 0, 1, 2, 3 or 4;
or a pharmaceutically acceptable salt thereof or a prodrug form that is metabolised to a compound as defined above.

The compounds of the invention may have an improved pharmacological profile compared to the parent compound. For example, the compounds may be better tolerated by the patient, or have an improved pharmacokinetic profile.

### DESCRIPTION OF THE INVENTION

The term “alkyl” as used herein refers to a straight or branched chain alkyl moiety having from one to six carbon atoms, and includes, for example, methyl, ethyl, propyl, isopropyl, butyl, tert-butyl, pentyl, hexyl and the like. “C₁₋₄ alkyl” has the same meaning.

The term “alkoxy” as used herein refers to a straight or branched chain alkoxy group containing one to six carbon atoms, and includes, for example, methoxy, ethoxy, propoxy, isopropoxy, butoxy, tert-butoxy, pentoxy, hexoxy and the like. “C₁₋₆ alkoxy” has the same meaning.

The term “halogen” as used herein refers to F, Cl, Br or I.

The term “heterocyclyl” as used herein refers to a saturated or unsaturated heterocyclic ring moiety having from four to seven carbon atoms and one or more heteroatoms selected from N. O, S. P and Si, and includes, for example, piperidinyl, pyrrolidinyl, morpholinyl and the like.

The term “alkanoyloxy” as used herein refers to a straight or branched chain alkanoyloxy moiety containing one to six carbon atoms.

The term “alkylmercapto” as used herein refers to a straight or branched chain alkylmercapto moiety containing one to six carbon atoms and includes, for example, methylmercapto.

The term “alkylamino” refers to a straight or branched chain alkylamino moiety containing one to six carbon atoms and includes, for example, methylamino.

The term “dialkylamino” refers to a dialkylamino moiety wherein each alkyl group is as defined above. This term includes, for example, dimethylamino.

The term “alkanamido” refers to a straightor branched chain alkanamido moiety containing two to six carbon atoms, and includes, for example, methanamido.

With regard to formula I, R¹ is preferably hydrogen or C₁₋₃ alkyl, more preferably methyl. R² is preferably C₁₋₃ alkyl, more preferably methyl. R¹ and R² may also form a heterocyclic ring, for example, NR¹R² may form a morpholinyl or piperidinyl group. R³ and R⁴ are preferably H or alkoxy. More preferably, R³ is hydrogen and R⁴ is methoxy. R⁵ is preferably hydrogen. It is also preferred that n is 0, 1 or 2. More preferably, n is 2.

Preferred compounds of the invention include:
1-[2-dimethylamino-1-(4-methoxyphenyl)ethyl]-1-silacyclohexan-1-ol;
1-[2-dimethylamino-1-(4-methoxyphenyl)ethyl]-1-silacyclobutan-1-ol; and
1-[2-dimethylamino-1-(4-methoxyphenyl)ethyl]-1-silacyclopentan-1-ol.

Compounds of the invention are chiral. They may be in the form of a single enantiomer or diastereomer, or a racemate.

The compounds of the invention may be prepared in racemic form, or prepared in individual enantiomeric form by specific synthesis or resolution as will be appreciated in the art. The compounds may, for example, be resolved into their enantiomers by standard techniques, such as the formation of diastereomeric pairs by salt formation with an optically active acid followed by fractional crystallisation and regeneration of the free base. Alternatively, the enantiomers of the novel compounds may be separated by HPLC using a chiral column.

The compounds of the invention may be in a protected amino form. The term “protected amino” as used herein refers to an amino group which is protected in a manner familiar to those skilled in the art. For example, an amino group can be protected by a benzyloxycarbonyl, tert-butoxycarbonyl, acetyl or like group, or in the form of a phthalimido or like group.

Some compounds of the formula may exist in the form of solvates, for example hydrates, which also fall within the scope of the present invention.

The compounds of the invention may exist in a prodrug form. In this aspect, the hydroxyl (OH) group attached to the silicon atom may comprise a group that is modified or removed under appropriate conditions to provide the compound in the active form. Suitable groups will be apparent to the skilled person, and include groups that replace the OH group on the silicon atom and which can be hydrolysed to form the OH group. For example, suitable replacement groups include H, OR⁶, N(R⁶)₂, or NHR⁶, where R⁶ is an alkyl group, preferably methyl. Hydrolysable phosphorus-containing or sulphur-containing groups may also be used in the prodrug forms.

Compounds of the invention may be in the form of pharmaceutically acceptable salts, for example, addition salts of inorganic or organic acids. Such inorganic acid addition salts include, for example, salts of hydrobromic acid, hydrochloric acid, nitric acid, phosphoric acid and sulphuric acid. Organic acid addition salts include, for example, salts of acetic acid, benzenesulphonic acid, benzoic acid, camphorsulphonic acid, citric acid, 2-(4-chlorophenoxy)-2-methylpropionic acid, 1,2-ethanedisulphonic acid, ethanesulphonic acid, ethylenediaminetetraacetic acid (EDTA), fumaric acid, glucoheptonic acid, gluconic acid, glutamic acid, N-glycolylarsanilic acid, 4-hexylresorcinol, hippuric acid, 2-(4-hydroxybenzoyl)benzoic acid, 1-hydroxy-2-naphthoic acid, 3-hydroxy-2-naphthoic acid, 2-hydroxyethanesulphonic acid, lactobionic acid, n-dodecyl sulphate, maleic acid, malic acid, mandelic acid, methanesulphonic acid, methyl sulphate, mucic acid, 2-naphthalenesulphonic acid, pamoic acid, pantothenic acid, phosphanilic acid ((4-aminophenyl)phosphonic acid), picric acid, salicyclic acid, stearic acid, succinic acid, tannic acid, tartaric acid, terephthalic acid, p-toluenesulphonic acid, 10-undecenoic acid and the like.

Salts may also be formed with inorganic bases. Such inorganic base salts include, for example, salts of aluminium, bismuth, calcium, lithium, magnesium, potassium, sodium, zinc and the like.

It will be appreciated that such salts, provided that they are pharmaceutically acceptable, may be used in therapy. Such salts may be prepared by reacting the compound with a suitable acid or base in a conventional manner.

A compound of the invention may be prepared by any suitable method known in the art and/or by the following processes shown in Schemes 1 and 2.

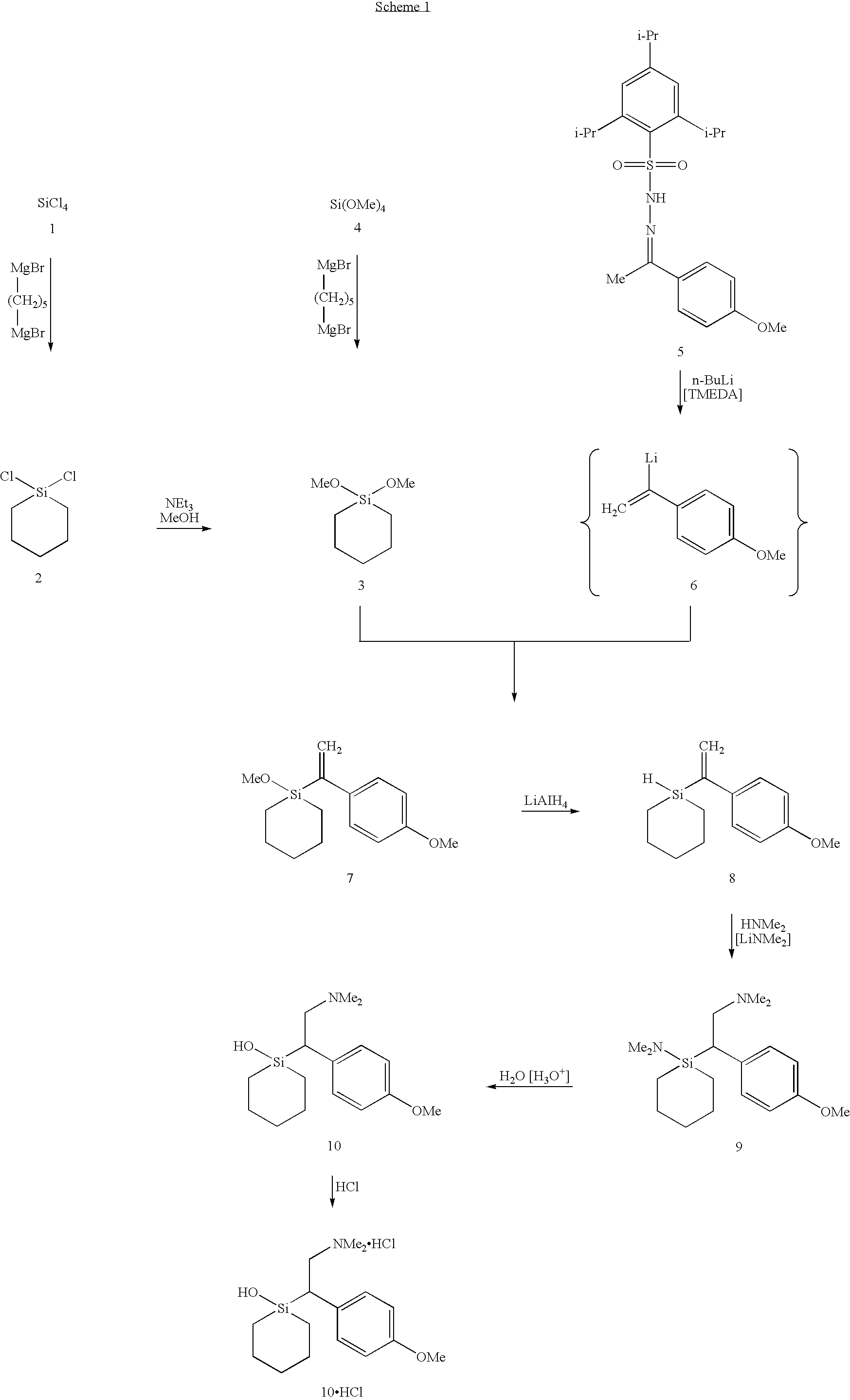

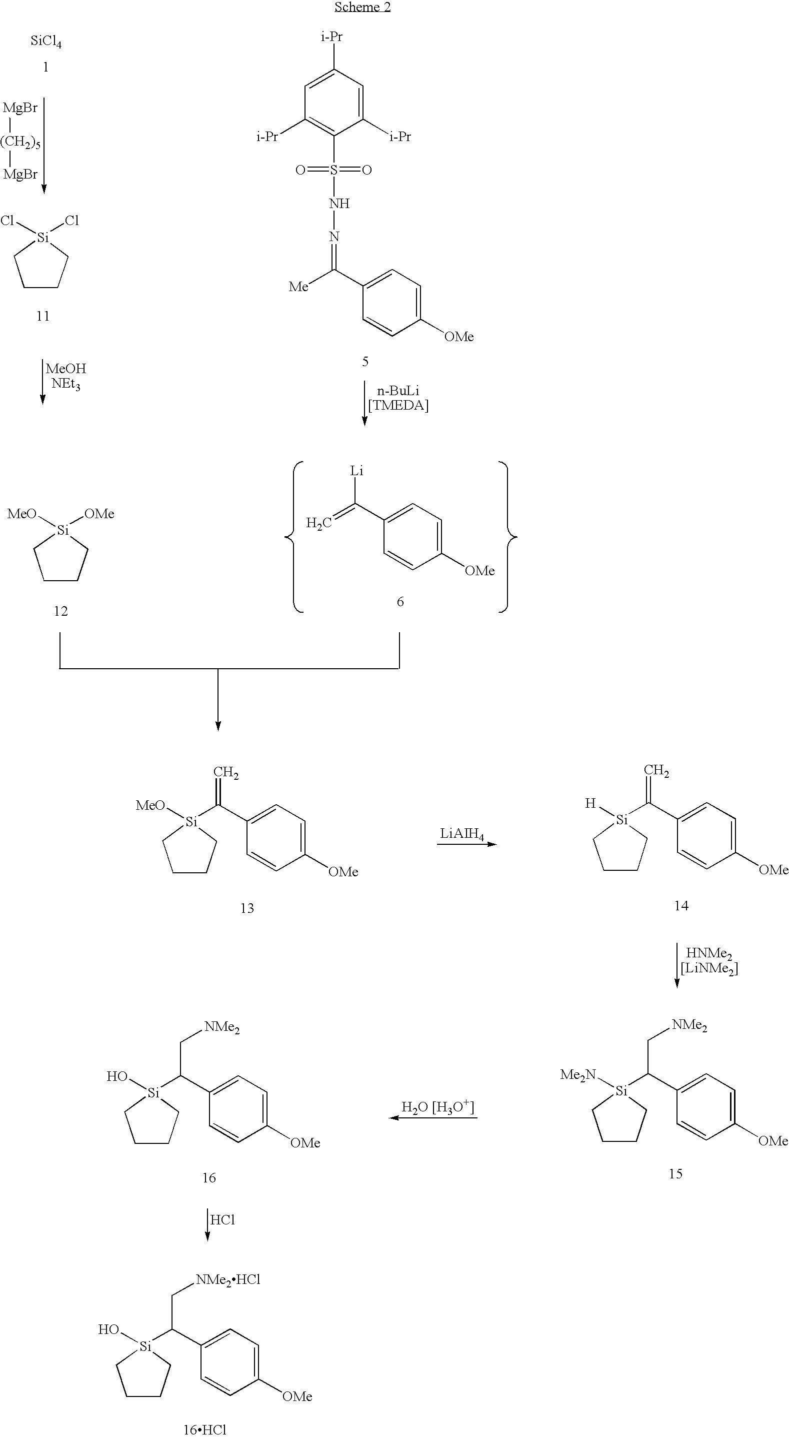

It will be understood that the processes detailed above are solely for the purpose of illustrating the invention and should not be construed as limiting. A process utilising similar or analogous reagents and/or conditions known to one skilled in the art may also be used to obtain a compound of the invention. In particular, compounds of the invention comprising alternatives to the groups R¹ to R⁵ illustrated in Schemes 1 and 2 may be synthesised by analogous processes, the alternative groups falling within the scope of formula I.

Any mixtures of final products or intermediates obtained can be separated on the basis of the physico-chemical differences of the constituents, in a known manner, into the pure final products or intermediates, for example by chromatography, distillation, fractional crystallisation, or by the formation of a salt if appropriate or possible under the circumstances.

The activity and selectivity of the compounds may be determined by any suitable assay known in the art.

As used herein, the term “active compound” denotes a compound of formula I including pharmaceutically acceptable salts thereof.

The compounds of the invention may be used in the treatment of numerous ailments, conditions and diseases including, but not limited thereto, addiction, anxiety, depression, sexual dysfunction, hypertension, migraine, Alzheimer's disease, obesity, emesis, psychosis, schizophrenia, Parkinson's disease, restless leg syndrome, sleeping disorders, attention deficit hyperactivity disorder, irritable bowel syndrome, premature ejaculation, menstrual dysphoria syndrome, premenstrual tension, urinary incontinence, pain, including inflammatory pain, neuropathic pain, chronic headache and chronic pain, Lesche-Nyhane disease, Wilson's disease and Tourette's syndrome.

In therapeutic use, the active compound may be administered orally, rectally, parenterally, by inhalation (pulmonary delivery), topically, ocularly, nasally, or to the buccal cavity. Oral administration is preferred. Thus, the therapeutic compositions of the present invention may take the form of any of the known pharmaceutical compositions for such methods of administration. The compositions may be formulated in a manner known to those skilled in the art so as to give a controlled release, for example rapid release or sustained release, of the compounds of the present invention. Pharmaceutically acceptable carriers suitable for use in such compositions are well known in the art. The compositions of the invention may contain 0.1-99% by weight of active compound. The compositions of the invention are generally prepared in unit dosage form. Preferably, a unit dose comprises the active ingredient in an amount of 1-500 mg. The excipients used in the preparation of these compositions are the excipients known in the art.

The administered dose is preferably similar to that of venlafaxine. For example, an initial dose may be 10-100 mg, 2-3 times daily or up to 150-400 mg daily in severely affected patients.

Appropriate dosage levels may be determined by any suitable method known to one skilled in the art. It will be understood, however, that the specific dose level for any particular patient will depend upon a variety of factors including the activity of the specific compound employed, the age, body weight, general health, sex, diet, time of administration, route of administration, rate of excretion, drug combination and the severity of the disease undergoing treatment.

Compositions for oral administration are preferred compositions of the invention and there are known pharmaceutical forms for such administration, for example tablets, capsules, granules, syrups and aqueous or oily suspensions. The pharmaceutical composition containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions, and such compositions may contain one or more agents selected from the group consisting of sweetening agents, flavouring agents, colouring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example corn starch or alginic acid; binding agents, for example starch gelatin, acacia, microcrystalline cellulose or polyvinyl pyrrolidone; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredient is mixed with an inert solid diluent, for example calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydroxypropylmethylcellulose, sodium alginate, polyvinyl pyrrolidone, gum tragacanth and gum acacia; dispersing or wetting agents may be a naturally occurring phosphatide, for example lecithin, or condensation products of an alkylene oxide with fatty acids, for example polyoxyethylene stearate, or condensation products of ethylene oxide with long-chain aliphatic alcohols, for example heptadecaethyleneoxycetanol, or condensation products of ethylene oxide with partial esters derived from fatty acids, for example polyoxyethylene sorbitan monooleate. The aqueous suspensions may also contain one or more preservatives, for example ethyl., or n-propyl, p-hydroxybenzoate, one or more colouring agents, one or more flavouring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents, suchas those set forth above, and flavouring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, suspending agent and one or more preservatives. Suitable sweetening, flavouring and colouring agents may also be present.

The pharmaceutical compositions of the invention may also be in the form of oil-in-water emulsions. The oily phase may be a vegetable oil, for example olive oil or arachis oil, dr a mineral oil, for example liquid paraffin, or mixtures of these. Suitable emulsifying agents may be naturally occurring gums, for example gum acacia or gum tragacanth, naturally occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavouring and colouring agents. The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be in a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid, find use in the preparation of injectables.

The compounds of formula I may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

Compositions for topical administration are also suitable for use in the invention. The pharmaceutically active compound may be dispersed in a pharmaceutically acceptable cream, ointment or gel. A suitable cream may be prepared by incorporating the active compound in a topical vehicle such as light liquid paraffin, dispersed in a aqueous medium using surfactants. An ointment may be prepared by mixing the active compound with a topical vehicle such as a mineral oil or wax. A gel may be prepared by mixing the active compound with a topical vehicle comprising a gelling agent. Topically administrable compositions may also comprise a matrix in which the pharmaceutically active compounds of the present invention are dispersed so that the compounds are held in contact with the skin in order to administer the compounds transdermally.

The following Examples illustrate the invention. Except for the conversions 9→10 and 15→16, all syntheses were carried out under dry nitrogen. Tetrahydrofuran, diethyl ether, methanol, triethylamine and n-hexane were dried and purified according to standard procedures and stored under nitrogen.

The compounds referenced numerically are those shown in Schemes 1 and 2, supra. Tetrachlorosilane (1) and tetramethoxysilane (4) are commercially available. With reference to the preparation of Intermediates 2, 3, 11 and 12, a similar method is disclosed by R. West, J. Am. Chem. Soc. 1954, 76, 6012-6014.

Intermediate 2: 1,1-Dichloro-1-silacyclohexane (2)

50 mL of a solution of 1,5-dibromopentane (161 g, 700 mmol) in diethyl ether (300 mL) was added to a stirred suspension of magnesium turnings (37.4 g, 1.54 mol) in diethyl ether (400 mL), and the reaction was started by gentle heating. Subsequently, the remaining 1,5-dibromopentane solution was added within 2 hours, causing the reaction mixture to boil under reflux. After the addition was complete, the mixture was heated under reflux for a further 90 min and was then allowed- to cool to 20° C. The resulting two-phase Grignard reagent (which was separated from residual magnesium turnings by decantation, followed by washing of the magnesium with diethyl ether (2×50 mL)) was added dropwise within 2 hours to a solution of 1 (131 g, 771 mmol) in diethyl ether (300 mL), causing the mixture to boil under reflux. During the addition, the mixture was stirred vigorously with a mechanical stirrer (precipitation of magnesium salts). The mixture was stirred for 16 hours at 20° C., and the precipitate was removed by filtration and washed with diethyl ether (2×200 mL). The filtrate and wash solutions were combined, and the solvent was removed by distillation under atmospheric pressure, causing a postprecipitation of magnesium salts. The precipitate was removed by decantation and washed with n-pentane (2×50 mL), and the organic solutions were combined. The solvent was removed as described above, and the crude product was isolated by distillation; bp 166-178° C./980 mbar. Redistillation (Vigreux column, 30 cm) under reduced pressure afforded 2 in 62% yield (related to 1,5-dibromopentane) as a colourless liquid (72.9 g, 431 mmol); bp 70-71° C./37 mbar.

Intermediate 3: 1,1-Dimethoxy-1-silacyclohexane (3)

Method A. Methanol (34.8 g, 1.09 mol) was added dropwise within 10 min to a stirred solution of 2 (83.2 g, 492 mmol) and triethylamine (110 g, 1.09 mol) in n-hexane (500 mL), causing the reaction mixture to boil under reflux (formation of a precipitate). After the addition was complete, the mixture was heated under reflux for a further 2 hours and was then allowed to cool to 20° C. and left undisturbed for 16 hours at this temperature. The precipitate was removed by suction filtration (700-750 mbar) using a Buchner funnel and washed thoroughly with n-hexane (1.5 L). The filtrate and wash solutions were combined, the solvent was removed by distillation under atmospheric pressure (Vigreux column, 20 cm), and the residue was distilled in vacuo (Vigreux column, 20 cm) to give 3 as a crude product (69 g; bp 70-75° C./30 mbar) that contained small amounts of a solid. The distillate was diluted with n-pentane (150 mL) and the mixture kept undisturbed at 4° C. for 16 hours, and the resulting precipitate was removed by filtration. The filter cake was washed with n-pentane (20 mL), and the filtrate and wash solution were combined. The solvent was removed by distillation under atmospheric pressure (Vigreux column, 30 cm) and the residue distilled in vacuo (Vigreux column, 30 cm) to give 3 in 80% yield as a colourless liquid (62.8 g, 392 mmol); bp 62° C./20 mbar.

Method B. A 1,5-bis(bromomagnesio)pentane reagent was prepared from magnesium turnings (22.0 g, 905 mmol), 1,5-dibromopentane (46.0 g, 200 mmol), and diethyl ether (200 mL) analogous to Method A (see above). The two-phase Grignard reagent was added at 0° C. over a period of 1 hour to a vigorously stirred solution of 4 (45.7 g, 300 mmol) in diethyl ether (500 mL) (formation of a precipitate). After the addition was complete, the mixture was heated under reflux for 16 hours and then allowed to cool to 20° C. The precipitate was removed by filtration and washed with diethyl ether (3×50 mL), the filtrate and wash solutions were combined, the solvent was removed under reduced pressure, and the residue was distilled twice in vacuo to give 3 in 43% yield (related to 1,5-dibromopentane) as a colourless liquid (13.9 g, 86.7 mmol); bp 75° C./36 mbar.

Intermediate 5: 4-Methoxyacetophenone 2,4,6-Triisopropylbenzenesulfonyl-hydrazone (5).

This compound was synthesised according to the general protocol described in Chamberlin et al, J. Org. Chem. 1978,43,147-154 (there referred to as Method A); see also Yu et al, Chem. Eur. J. 1997, 3, 417-423.

Intermediates 6 and 7: 1-(4-Methoxyphenyl)vinyllithium (6) and 1-Methoxy-1-[1-(4-methoxyphenyl)vinyl]-1-silacyclohexane (7).

A 2.7 M solution of n-butyllithium in n-heptane (70 mL, 189 mmol of n-BuLi) was added dropwise at −78° C. within 50 min to a stirred mixture consisting of 5 (40.0 g, 92.9 mmol), N,N,N′,N′-tetramethylethylenediamine (40 mL), and n-hexane (360 mL). The resulting yellow mixture was stirred at −78° C. for 2 hours and then allowed to warm to 0° C. (evolution of nitrogen; change of colour to orange; formation of 1-(4-methoxyphenyl)vinyllithium (6)). After the nitrogen evolution was finished, the mixture was stirred for a further 10 min at 20° C. and then added dropwise at 0° C. within 30 min to a solution of 3 (15.0 g, 93.6 mmol) in n-hexane (100 mL). The resulting mixture was allowed to warm to 20° C. (change of colour from orange to yellow within ca. 12 hours) and stirred for 3 days. The resulting clear yellow solution was cooled in an ice bath, and iodomethane (125 g, 881 mmol) was added (formation of a precipitate). After a period of 2 hours, the ice bath was removed and stirring was continued for 1 day at 20° C. The precipitate was removed by filtration and washed with n-hexane (4×250 mL), and the filtrate and wash solutions were combined. The solvent was removed under reduced pressure (300 mbar, 40° C.; rotary evaporator) and the residue distilled in vacuo (Kugelrohr apparatus; first fraction: <90° C./0.001 mbar, discarded; second fraction: 90-145° C./0.001 mbar, crude product). The crude products of three identical runs of this preparation were combined (43.0 g) and distilled in vacuo (Vigreux column, 15 cm) to give 7 in 45% yield (related to 3) as a colourless oily liquid (33.2 g, 127 mmol); bp 105° C./0.001 mbar.

Intermediate 8: 1-[1-(4-Methoxyphenyl)vinyl]-1-silacyclohexane (8).

A solution of 7 (32.0 g, 122 mmol) in diethyl ether (50 mL) was added at 20° C. within 10 min to a stirred suspension of lithium aluminium hydride (2.48 g, 65.3 mmol) in diethyl ether (200 mL). The mixture was heated under reflux for 2 hours, allowed to cool to 20° C., and then added slowly at 0° C. to a stirred mixture of 4 M hydrochloric acid (210 mL) and diethyl ether (100 mL). The organic phase was separated and the aqueous layer extracted with diethyl ether (3×100 mL). The combined organic solutions were dried over anhydrous magnesium sulphate in an ice bath, followed by an additional thorough dynamic drying using a chromatographic column densely packed with anhydrous magnesium sulphate (column diameter, 3.5 cm; column length, 15 cm). The magnesium sulphate was finally washed with diethyl ether (500 mL), and the organic solutions were combined. The solvent was removed at 800-900 mbar (rotary evaporator) and the residue distilled in vacuo (Vigreux column, 15 cm) to give 8 in 82% yield as a colourless oily liquid (23.3 g, 100 mmol); bp 91-92° C./0.001 mbar.

Intermediate 9: 1-Dimethylamino-1-[2-dimethylamino 1-(4-methoxyphenyl)ethyl]-1-silacyclohexane (9).

A 1.6 M solution of n-butyllithium in n-hexane (9.5 mL, 15.2 mmol of n-BuLi) was added dropwise at −50° C. within 5 min to a stirred solution of dimethylamine (5.51 g, 122 mmol) in tetrahydrofuran (150 mL). The resulting mixture was allowed to warm to −15° C. within 4 hours and was then cooled to −35° C., followed by dropwise addition of 8 (3.20 g, 13.8 mmol) within a period of 10 min (evolution of hydrogen; rise in temperature from ° C. to −30° C.). The resulting yellow solution was stirred at −30° C. for 3 hours and was then kept undisturbed at −26° C. for 16 hours. Subsequently, the solution was placed in an ice bath and stirred again, followed by addition of chlorotrimethylsilane (1.72 g, 15.8 mmol) in one single portion (change of colour from yellow to colourless). The mixture was stirred at 0° C. for 30 min, and the solvent was removed completely in vacuo in a water bath (5-15° C.), followed by addition of n-hexane (40 mL). The mixture was stirred for 30 min at 20° C., the resulting precipitate was removed by filtration, and the filter cake was washed with n-hexane (20 mL). The filtrate and the wash solution were combined, and the solvent was removed completely in vacuo in a water bath (5-15° C.) and the residue distilled in vacuo (Vigreux column, 5 cm) to give 9 in 76% yield as a colourless oily liquid (3.37 g, 10.5 mmol); bp 115-118° C./0.003 mbar.

Intermediate 11: 1,1-Dichloro-1-silacyclopentane (11).

This compound was prepared analogously to the synthesis of 2 (1,4-dibromobutane (151 g, 699 mmol), magnesium turnings (37.4 g, 1-0.54 mol), 1 (131 g, 771 mmol)). After distillation under atmospheric pressure (Vigreux column, 15 cm; 71 g of crude product; bp 141-145° C.) and redistillation in vacuo (Vigreux column, 30 cm), compound 11 was isolated in 61% yield (related to 1,4-dibromobutane) as a colourless liquid (66.29 g, 427 mmol); bp 71-73° C./100 mbar.

Intermediate 12: 1,1-Dimethoxy-1-silacyclopentane (12).

This compound was prepared analogously to the synthesis of 3, method A (11 (66.2 g, 427 mmol), methanol (30.4 g, 949 mmol), triethylamine (96.1 g, 950 mmol)). After distillation under atmospheric pressure (Vigreux column, 15 cm; 53 g of crude product; bp 136-144° C.) and redistillation in vacuo, compound 12 was isolated in 74% yield as a colourless liquid (46.2 g, 316 mmol); bp 73° C./100 mbar.

Intermediate 13: 1-Methoxy-1-[1-(4-methoxyphenyl)vinyl]-1-silacyclopentane (13).

A 2.7 M solution of n-butyllithium in n-heptane (70 mL, 189 mmol of n-BuLi) was added dropwise at −78° C. within 50 min to a stirred mixture consisting of 5 (40.0 g, 92.9 mmol), N,N,N′,N′-tetramethylethylenediamine (40 mL), and n-hexane (360 mL). The resulting yellow mixture was stirred at −78° C. for 2 hours and then allowed to warm to 0° C. (evolution of nitrogen; change of colour to orange; formation of 1-(4-methoxyphenyl)vinyllithium (6)). After the nitrogen evolution was finished, the mixture was stirred for a further 10 min at 20° C. and then added dropwise at −55+5° C. within 30 min to a solution of 12 (14.3 g, 97.8 mmol) in n-hexane (200 mL). The resulting mixture was allowed to warm to −30° C. within 2 hours and then to 10° C. within a further 15 hours, and was finally stirred at 20° C. for 1 day. The resulting clear yellow solution was cooled in an ice bath, and iodomethane (125 g, 881 mmol) was added (formation of a precipitate). After a period of 2 hours, the ice bath was removed and stirring was continued for 1 day at 20° C. The precipitate was removed by filtration and washed with n-hexane (4×250 mL), and the filtrate and wash solutions were combined. The solvent was removed under reduced pressure (300 mbar, 40° C.; rotary evaporator) and the residue distilled in vacuo (Kugelrohr apparatus; first fraction: ≦90° C./0.001 mbar, discarded; second fraction: 90-140° C./0.001 mbar, crude product; 15.8 g). Distillation in vacuo (Vigreuxcolumn, 15 cm) gave 13 in 45% yield (related to 12) as a colourless oily liquid (10.9 g, 43.9 mmol); bp 90° C./0.001 mbar.

Intermediate 14: 1-[1-(4-Methoxyphenyl)vinyl]-1-silacyclopentane (14).

This compound was prepared analogously to the synthesis of 8 (13 (10.7 g, 43.1 mmol), lithium aluminium hydride (820 mg, 21.6 mmol), diethyl ether (100 mL)) and was isolated in 79% yield as a colourless oily liquid (7.45 g, 34.1 mmol); bp 77° C./0.001 mbar.

Intermediate 15: 1-Dimethylamino-[2-dimethylamino-1-(4-methoxyphenyl)ethyl]-1-silacyclopentane (15).

This compound was prepared analogously to the synthesis of 9 (14 (2.52 g, 11.5 mmol), dimethylamine (7.07 g, 157 mmol), a 1.6 M solution of n-butyllithium in n-hexane (7.9 mL, 12.6 mmol of n-BuLi), chlorotrimethylsilane (1.46 g, 13.4 mmol), tetrahydrofuran (45 mL)) and was isolated in 60% yield as a colourless oily liquid (2.13 g, 6.95 mmol); bp 112-113° C./0.001 mbar.

### EXAMPLE 1

### 1-[2-Dimethylamino-1-(4-methoxyphenyl)ethyl]-1-silacyclohexan-1-01 (Sila-venlafaxine, 10; identical with (±)-10).

A 2.7 M solution of n-butyllithium in n-heptane (35 mL, 94.5 mmol of n-BuLi) was added dropwise at −50° C. within 10 min to a stirred solution of dimethylamine (21.6 g, 479 mmol) in tetrahydrofuran (100 mL). The resulting mixture was allowed to warm to −10° C. within 2 hours and was then cooled to −40° C., followed by dropwise addition of 8 (20.0 g, 86.1 mmol) within a period of 15 min (evolution of hydrogen; rise in temperature from −40° C. to −35° C.). The resulting stirred yellow solution was allowed to warm to −20° C. within 2 hours and then kept undisturbed at −26° C. for 16 hours. Subsequently, the solution was allowed to warm to 20° C., and the solvent was removed in vacuo in a water bath (5-15° C.) until a residual volume of 50 mL was obtained. This solution was diluted with diethyl ether (200 mL) and then added in one single portion at 0° C. to a stirred two-phase mixture of diethyl ether (50 mL) and 2 M potassium acetate/acetic acid buffer (pH 4.5, 300 mL). The pH of the aqueous phase changed to pH 7.2 within 10 min and was readjusted to pH 5.0 by adding small portions of glacial acetic acid. The mixture was stirred for a further 1 hour at 0° C., with the pH of the aqueous phase remaining constantly at pH 5.0 during this time. The aqueous layer was separated and the organic phase extracted with 1 M potassium acetate/acetic acid buffer (pH 5.0), and the aqueous solutions were combined. Diethyl ether (150 mL) was added, and the pH of the aqueous phase was adjusted to pH 10.5 by adding small portions of saturated aqueous potassium carbonate solution. The organic layer was separated and the aqueous phase extracted with diethyl ether (5×100 mL). The organic extracts were combined, followed by addition of n-hexane (200 mL). The solvent was removed in vacuo in a water bath (5-15° C.) until a residual volume of 100 mL was obtained, whereupon residual water separated from the organic phase (formation of a two-phase system). The organic layer was separated, the aqueous phase was extracted with n-hexane (2×100 mL), and the organic solutions were combined. The solvent was removed completely in vacuo in a water bath (5-15° C.) to give a colourless oil. Crystallisation of this oil from n-pentane (400 mL) at −26° C. using seed crystals (obtained by cooling of a solution of oily 10 (3.20 g) in n-pentane (5 mL) to −26° C.) afforded 10 in 90% yield as a colourless crystalline solid (22.8 g, 77.7 mmol) (isolated by quick decantation of the cold solvent, followed by drying in vacuo (0.001 mbar, 20° C., 6 hours)); mp 33° C.

### EXAMPLE 2

### (−)-1-[2-Dimethylamino-1-(4-methoxyphenyl)ethyl]-1-silacyclohexan-1-ol ((−)-Sila-venlafaxine, (−)-10).

(a) Seed Crystals of (−)-Sila-venlafaxine(+)-10-Camphorsulphonic Acid ((−)-10·(+)-CSA).

A solution of (+)-10-camphorsulphonic acid ((+)-CSA) (792 mg, 3.41 mmol) in acetone (25 mL) was added at 0° C. to a solution of (±)-10 (1.00 g, 3.41 mmol) in acetone (25 mL). After the mixture was shaken briefly, it was kept undisturbed at 0° C. After ca. 10 min, thin needle-shaped crystals precipitated. A further 40 mL of acetone was added immediately, and the mixture was then kept undisturbed at 4° C. for 2 days. The precipitate was isolated by filtration, washed with acetone (20 mL), and recrystallised twice from boiling acetone (45 mL). (To leave a few seed crystals, the solid was not allowed to dissolve completely in both recrystallisation steps). The product was finally isolated by filtration, washed with acetone (3 mL), and dried in vacuo (0.001 mbar, 20° C., 6 hours) to give 629 mg of: a colourless crystalline solid. This material (long, very thin needles) was used as seed crystals in the following protocol.

(b) (−)-Sila-venlafaxine-(+)-10-Camphorsulphonic Acid ((−)-10·(+)-CSA).

A solution of (+)-CSA (4.55 g, 19.6 mmol) in acetone (120 mL) was added at 20° C. to a solution of (±)-10 (5.75 g, 19.6 mmol) in acetone (375 mL). After the mixture was shaken briefly, it was kept undisturbed at 4° C. for 2 hours. After a few seed crystals (see above) were added, the mixture was kept undisturbed at 4° C. for 2 days. The resulting precipitate was isolated by filtration, washed with acetone (2×20 mL), and then recrystallised twice from boiling acetone (280 mL; crystallisation at 4° C., 2 days). (To leave a few seed crystals, the solid was not allowed to dissolve completely in these recrystallisation steps). The product was isolated and washed as described above and finally dried in vacuo (0.001 mbar, 20° C., 6 hours) to give (−)-10(+)-CSA in 30% yield (related to (±)-10) as a colourless crystalline solid (3.10 g, 5.90 mmol); mp 164° C.

(c) (−)-Sila-venlafaxine ((−)-10).

Diethyl ether (5 mL) was added at 20° C. to a stirred solution of (−)-10·(+)-CSA (3.05 g, 5.80 mmol) in water (85 mL), and the pH of the aqueous phase was adjusted to pH 10.5 by addition of saturated aqueous potassium carbonate solution. The resulting mixture was extracted with diethyl ether (4×100 mL) and the organic layers were combined, followed by addition of n-hexane (200 mL). The solvent was removed in vacuo in a water bath (5-15° C.) until a residual volume of 50 mL was obtained. The mixture was then kept at −20° C. for 3 hours (crystallisation of the residual water), and the organic supernatant was quickly isolated by decantation and stored separately. The ice was allowed to melt, the resulting aqueous phase was shaken with n-hexane (60 mL), and the two-phase system was again kept at −20° C. for 3 hours. The decantation procedure was repeated, the organic solutions were combined, and the solvent was removed in vacuo in a water bath (5-15° C.). The resulting colourless oil was dissolved in n-pentane (35 mL) and the solution kept undisturbed at −20° C. After a period of ca. 2-3 hours, an oil separated, and a few crystals grew within the oil drops. The mixture was then allowed to warm to 20° C., whereupon the oil dissolved rapidly, whereas the crystals dissolved only slowly. After most of the crystals were dissolved (except for a few seed crystals), the mixture was again kept undisturbed at −20° C. for 3 days. The resulting crystalline product was isolated by decantation and then dried in vacuo (0.001 mbar, 20° C., 6 hours) to give (−)-10 in 99% yield as a colourless crystalline solid (1.68 g, 5.72 mmol; including workup of the mother liquour by concentration to 10 mL and using the crystallisation protocol described above); mp 64-65° C.

### EXAMPLE 3

### (+)-1-[2-Dimethylamino-1-(4-methoxyphenyl)ethyl]-1-silacyclohexan-1-ol ((+)-Sila-venlafaxine, (+)-10).

The combined mother liquors obtained in the preparation of (−)-10·(+)-CSA (see above) were used to prepare (+)-10. For this purpose, the mother liquors were concentrated in vacuo, treated with potassium carbonate as described for the preparation of (−)-10, concentrated again, and the oily residue was then treated with (−)-CSA as described above. (a) (+)-10(−)-CSA. Yield 32% (related to (±)-10) of a colourless crystalline solid (3.29 g, 6.26 mmol); mp 164° C. (b) (+)-10. Prepared from (+)-10·(−)-CSA (3.23 g, 6.14 mmol); yield: 94% of a colourless crystalline solid (1.70 g, 5.79 mmol); mp 64-65° C.

### EXAMPLE 4

### [2-(1-Hydroxy-1-silacyclohexan-1-yl)-2-(4-methoxyphenyl)ethyl]dimethylammonium Chloride (Sila-venlafaxine Hydrochloride, 10·HCl).

A 2 M ethereal HCl solution (23 mL, 46.0 mmol of HCl) was added in one single portion at 20° C. to a stirred solution of 10 (12.9 g, 44.0 mmol) in dichloromethane (200 mL). The resulting solution was cooled to −11° C., and a few seed crystals (obtained from 20 μL of the reaction mixture by slow evaporation of the solvent at 20° C.) were added. The mixture was kept undisturbed for 1 day at −11° C. and then for a further 1 day at −27° C. The solid was isolated by filtration at −27° C., washed with ice-cold acetone (20 mL), and dried in vacuo (0.001 mbar, 20° C., 6 hours) to give 10·HCl in 90% yield (including workup of the mother liquour) as a colourless crystalline solid; (13.0 g, 39.4 mmol); mp 160° C.

### EXAMPLE 5

### (−)-[2-(1-Hydroxy-1-silacyclohexan-1-yl)-2-(4-methoxyphenyl)ethyl]dimethyl-ammonium Chloride ((−)-Sila-venlafaxine Hydrochloride, (−)-10·HCl).

A 2 M ethereal HCl solution (1.8 mL, 3.6 mmol of HCl) was added at 20° C. to a solution of (−)-10 (1.00 g, 3.41 mmol) in dichloromethane (19 mL), and the resulting mixture was shaken briefly. Upon vapour diffusion of diethyl ether into this mixture at 20° C. for 6 days, a crystalline product was obtained, which was isolated by filtration, washed with diethyl ether (40 mL), and finally dried in vacuo (0.001 mbar, 20° C., 6 hours) to give (−)-10·HCl in 93% yield as a colourless crystalline solid (1.04 g, 3;15 mmol); mp 174° C.

### EXAMPLE 6

### (+)-[2-(1-Hydroxy-1-silacyclohexan-1-yl)-2-(4-methoxyphenyl)ethyl]dimethyl-ammonium Chloride ((+)-Sila-venlafaxine Hydrochloride, (+)-10·HCl).

This compound was prepared from (+)-10 (1.00 g, 3.41 mmol) analogous to the protocol used for the preparation of (−)-10·HCl and isolated in 92% yield as a colourless crystalline solid (1.03 g, 3.12 mmol); mp 174° C.

### EXAMPLE 7

### 1-[2-Dimethylamino-1-(4-methoxyphenyl)ethyl]-1-silacyclopentan-1-61 (16).

This compound was prepared analogously to the synthesis of 10 (14 (2.54 g, 11.6 mmol), dimethylamine (8.07 g, 179 mmol), a 1.6 M solution of n-butyllithium in n hexane (8.0 mL, 12.8 mmol of n-BuLi), tetrahydrofuran (65 mL)). The oily crude product crystallized from n-pentane (45 mL; −11° C. (1 hour)→26° C.(1 day)), and compound 16 was isolated in 54% yield as a colourless crystalline solid; (1.77 g, 6.33 mmol); mp 37° C.

### EXAMPLE 8

### [2-(1-Hydroxy-1-silacyclopentan-1-yl)-2-(4-methoxyphenyl)ethyl]-dimethylammonium Chloride (16·HCl).

A 2 M ethereal HCl solution (2.0 mL, 4.0 mmol of HCl) was added at 20° C. in one single portion to a stirred solution of 16 (1.02 g, 3.65 mmol) in dichloromethane (16 mL). The mixture was kept undisturbed at −27° C. for 2 hours, and a few seed crystals (obtained from 20 μL of the reaction mixture by slow evaporation of the solvent at 20° C., followed by cooling of the resulting oil to −27° C.) were added. The resulting mixture was kept undisturbed at −27° C. for three days, and the precipitate was isolated by filtration at −27° C., washed with ice-cold acetone (10 mL), and then dried in vacuo (0.001 mbar, 20° C., 6 hours) to give 16·HCl in 52% yield as a colourless crystalline solid (598 mg, 1.89 mmol); mp 153-154° C.

1. A compound of formula I: 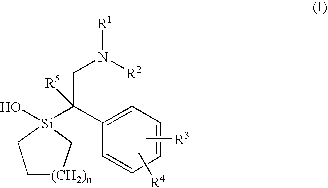
wherein R¹ and R² are, independently, hydrogen or alkyl or together, with the nitrogen atom, are heterocyclyl;
R³ and R⁴ are, independently, hydrogen, hydroxyl, alkyl, alkoxy, alkanoyloxy, cyano, nitro, alkylmercapto, amino, alkylkamino, dialkylamino, alkanamido, halogen or trifluoromethyl;
R⁵ is hydrogen or alkyl; and
n is 0, 1, 2, 3 or 4;
or a pharmaceutically acceptable salt thereof or a prodrug form that is hydrolysable to a compound as defined above.

2. The compound according to claim 1, wherein R¹ and R² are, independently, hydrogen or C₁₋₃ alkyl.

3. The compound according to claim 2, wherein R¹ and R² are the same or different and are each C₁₋₃ alkyl.

4. The compound according to claim 1, wherein R³ is hydrogen and R⁴ is alkoxy.

5. The compound according to claim 4, wherein R⁴ is methoxy.

6. The compound according to claim 1, wherein n is 0, 1 or 2.

7. The compound according to claim 6, wherein n is 2.

8. The compound according to claim 1, wherein R⁵ is hydrogen.

9. The compound according to claim 1, selected from the group consisting of:
1-[2-dimethylamino-1-(4-methoxyphenyl)ethyl]-1-silacyclobutan-1-ol; and 1-[2-dimethylamino-1-(4-methoxyphenyl)ethyl]-1-silacyclopentan-1-ol; in either racemic or enantiomeric form.

10. The compound according to claim 9, selected from the group consisting of:
(−)-1-[2-dimethylamino-1-(4-methoxyphenyl)ethyl]-1-silacyclohexan-1-ol; and (+)-1-[2-dimethylamino-1-(4-methoxyphenyl)ethyl]-1-silacyclohexan-1-ol.

11. (cancel).

12. A pharmaceutical composition comprising a compound of formula I: 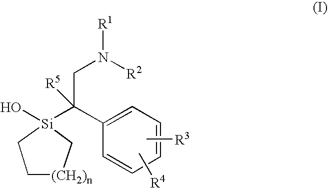
wherein R¹ and R² are, independently, hydrogen or alkyl or together, with the nitrogen atom, are heterocyclyl;
R³ and R⁴ are, independently, hydrogen, hydroxyl, alkyl, alkoxy, alkanoyloxy, cyano, nitro, alkylmercapto, amino, alkylkamino, dialkylamino, alkanamido, halogen or trifluoromethyl:
R⁵ is hydrogen or alkyl: and
n is 0, 1.2, 3 or 4,
or a pharmaceutically acceptable salt thereof or a prodrug form that is
hydrolysable to a compound as defined above together with a pharmaceutically acceptable carrier or diluent.

13. A method for the treatment or prevention of addiction, anxiety, depression, sexual dysfunction, hypertension, migraine, Alzheimer's disease, obesity, emesis, psychosis, schizophrenia, Parkinson's disease, restless leg syndrome, sleeping disorders, attention deficit hyperactivity disorder, irritable bowel syndrome, premature ejaculation, menstrual dysphoria syndrome, premenstrual tension, urinary incontinence, pain, including inflammatory pain, neuropathic pain, chronic headache and chronic pain, Lesche-Nyhane disease, Wilson's disease or Tourette's syndrome wherein said method comprises administering to a patient in need of such treatment a compound of formula I: 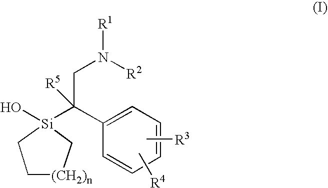
wherein R¹ and R² are, independently, hydrogen or alkyl or together with the nitrogen atom, are heterocyclyl:
R¹ and R⁴ are, independenly, hydroxyl, alkyl, alkoxy, alkanoyloxy, cyano, nitro, alkylmercapto, amino, alkylkamino, dialkylamino, alkanamido, halogen or trifluoromethyl;
R⁵ is hydrogen or alkyl; and
n is 0, 1, 2, 3 or 4;
or a pharmaceutically acceptable salt thereof or a for a prodrug form that is hydrolysable to a compound as defined above.
